(19) United States
(12) Patent Application Publication
(10) Pub. No.: US 2021/0222208 A1
(43) Pub. Date: Jul. 22, 2021

(54) SUGAR-DERIVED STIMULANT FOR ENHANCEMENT OF AEROBIC AND ANAEROBIC FERMENTATION PERFORMANCE

(71) Applicant: THE REGENTS OF THE UNIVERSITY OF CALIFORNIA, Oakland, CA (US)

(72) Inventors: Charles E. WYMAN, Riverside, CA (US); Abhishek S. PATRI, West Sacramento, CA (US); Charles CAI, Riverside, CA (US); Rajeev KUMAR, Ahmedabad, Gujarat (IN); Priyanka SINGH, Riverside, CA (US)

(73) Assignee: THE REGENTS OF THE UNIVERSITY OF CALIFORNIA, Oakland, CA (US)

(21) Appl. No.: 17/256,053

(22) PCT Filed: Jun. 27, 2019
(86) PCT No.: PCT/US2019/039480

§ 371 Date: Dec. 24, 2020

Related U.S. Application Data
(60) Provisional application No. 62/690,808, filed on Jun. 27, 2018.

(51) Int. Cl. C12P 7/10; C12N 1/16; C07H 15/04; C12N 1/20

(52) U.S. Cl. CPC C12P 7/10; C12N 1/20; C07H 15/04; C12N 1/16

(57) ABSTRACT

Described herein are microbial stimulating compounds that act as a surfactant to increase fermentation. Also described are methods for enhancing fermentation utilizing these compounds as well as methods for the producing the compounds from lignocellulosic biomass and biomass components during high temperature reactions with alcohols. The stimulating compounds can be produced from a variety of polysaccharides or sugars.

### CROSS REFERENCE TO RELATED APPLICATIONS

This application is a National Stage filing under 35 U.S.C. 371 of International Application No. PCT/US2019/039480, filed on Jun. 27, 2019, which claims the benefit of U.S. Provisional Application 62/690,808, filed Jun. 27, 2018, the entire content of both of which is incorporated herein.

### STATEMENT OF GOVERNMENT INTERESTS

This invention was made with government support under Contract No. DE-PS0206ER64304 and Grant No. 0577501-18-0029 awarded by the United States Department of Energy (DOE). The Government has certain rights in the invention.

### FIELD

The disclosure is related to compositions that enhance fermentation processes, such as, for example, biomass conversion into ethanol, and methods of making the aforementioned compositions.

### BACKGROUND

In the energy field, biofuels provide an attractive substitute for conventional fossil fuels due to the use of renewable feedstocks that reduce greenhouse gas emissions and foreign resource dependence. Pretreatments are typically required prior to biological conversions to deconstruct plant cell wall structures to sugars with high yields. Acid-based thermochemical pretreatments have been historically studied to solubilize hemicellulose and disrupt the cell wall structure to increase access to cellulose. The liquid hydrolyzate after acid pretreatments, including dilute sulfuric acid (DSA) pretreatment, a current research and commercial benchmark, and Co-solvent Enhanced Lignocellulosic Fractionation (CELF), an advanced pretreatment, typically contains a large portion of the hemicellulose sugars, often composed mostly of xylose; fermentation organisms have been genetically engineered to ferment these pentose sugars in addition to natively-fermented glucose that is released during hydrolysis of cellulose in pretreated biomass to ethanol with high yields; however, effective xylose utilization remains a challenge, with slow xylose metabolic rates reducing overall ethanol productivity in fermentation processes.

Pretreatment hydrolyzates can comprise monomeric hemicellulose sugars, amenable to fermentation to ethanol, along with acetic acid, THF, 1,4-butanediol (BDO), and lignin-derived phenolics. Phenolics have been shown to inhibit fermenting microorganisms. While not wanted to be limited by theory, these compounds are thought to penetrate into the membranes of the microorganisms and cause a loss of integrity, thus affecting the cell's ability to effectively transport substrates and products. In addition, THF has also been shown to be inhibitory to microorganisms.

Removal of fermentation inhibitors from pretreatment hydrolyzates has been by adsorptive materials, biodegradation, and solvent extraction.

The conversion of sugars to ethanol at high yields and rates is critical to making commercial fermentation processes competitive.

Typically, fermentations are started with an aerobic seed flask where cell mass is increased using a sugar source and then transferred to an anaerobic fermenter where sugars are converted by microorganisms such as Saccharomyces cerevisiae to ethanol or other products. Rates of growth and fermentation performance are correlated to ethanol yield; any supplement or enhancement of the fermentation to increase growth rate or fermentation performance is highly desirable.

Saccharomyces cerevisiae is a commercial benchmark microorganism used in sugar fermentations. The yeast S. cerevisiae has been utilized for centuries for anaerobic fermentation of sugars to ethanol with high yields and relatively high concentrations and remains the primary organism for commercial ethanol production. Its high ethanol tolerance, ability to grow under strictly anaerobic conditions, and low pH tolerance contribute to it being ideal for commercial fermentations. Since the S. cerevisiae genome has been completely sequenced, selective modification of the organism's genes is relatively straightforward. However, although S. cerevisiae rapidly ferments hexose sugars, such as glucose, fructose, and mannose, it is unable to anaerobically metabolize pentose sugars, such as xylose and arabinose, with its native genome. This limitation is of particular significance as the majority of sugars solubilized by acid and some other pretreatments of lignocellulosic biomass to make it accessible for enzymatic deconstruction are pentose sugars whose conversion to ethanol is crucial to cost effective processing of biomass to fuels. Although, S. cerevisiae strains have been engineered to ferment pentose sugars to ethanol, xylose consumption lags glucose metabolism due to the diauxic effect that slows fermentations and can result in lower yields from pentose sugars. Nevertheless, strains of S. cerevisiae have been the industry standard for choice of fermentation microorganisms for decades.

As with all commercial processes, an improvement in overall annual productivity is highly desirable. To do so, various additives, such as non-ionic surfactants, Tween 20 and Tween 80, have been reported to enhance fermentation rates of sugars to ethanol. Additionally, non-aryl, non-ionic surfactants such as polyethylene glycol (PEG), methoxy polyethylene glycol (MPEG), dimethoxy polyethylene glycol (DMPEG), and polydimethylsiloxane (PDMS) have been implemented as to increase cell viability and aid in high gravity ethanol fermentations. However, among non-ionic surfactants, the overall effect on ethanol fermentation is greatly dependent on the surfactant, with Tween 20 and Tween 80 having slightly positive impact on fermentation rates/yields but with Triton X-100 having a negative effect.

In addition, surfactants, such as Tween 80, have been demonstrated to stimulate microorganisms and product formation. However, even with Tween 80, it is thought that xylose, galactose, and arabinose could not be effectively utilized by S. cerevisiae with Wei et al. only reporting the uptake of glucose and mannose.

Alkyl polyglycosides are non-ionic surfactants that are applied industrially and can be produced by Fischer glycosidation of glucose with an alcohol in the presence of an acid catalyst. Alkyl polyglycosides have been reported to stimulate anaerobic fermentations of food waste. Additionally, the production of alkyl polyglycosides from glucose released by cellulose hydrolysis has been investigated.

As a result, improvements in performance of existing fermentation processes and novel methods to increase xylose fermentation rates and enhance xylose conversions so that overall fermentation efficiency can be enhanced are highly coveted.

### SUMMARY

Some embodiments describe an enhanced fermentation mixture. Some mixtures can comprise a sugar-containing fermentation precursor and stimulant. In some embodiments, the stimulant can be a hydroxy-C₃₋₈ alkyl glucopyranoside. In some mixtures, the hydroxy-C₃₋₈ alkyl glucopyranoside can comprise a 4-hydroxybutyl glucopyranoside. In some embodiments, the sugar-containing fermentation precursor can comprise a lignocellulose biomass. For some mixtures, the sugar-containing fermentation precursor can comprise a glucose and/or a xylose. In some embodiments, the mass ratio of xylose to glucose can vary from about 0 to about 7. In another embodiment, the sugar-containing fermentation precursor is a xylose.

Some embodiments depict a method of improving fermentation, where the method can comprise: obtaining a fermentation mixture, adding a fermentation stimulant to the fermentation mixture before fermentation is commenced, and proceeding with fermentation, where adding the fermentation stimulant can comprise adding a glucoside to the fermentation mixture. In some embodiments, the step of adding glucoside can comprise adding hydroxy-C₃₋₈ alkyl glucopyranoside to the fermentation mixture. In some methods, the step of adding glucoside can comprise adding 4-hydroxybutyl glucopyranoside to the fermentation mixture. For some methods, adding the fermentation stimulant can comprise adding between 0.1% vol. to 35% vol. of the stimulant to the mixture. For some embodiments, adding the fermentation stimulant can comprise adding 2% vol. of the stimulant to the mixture.

Some embodiments characterize a method of making a fermentation stimulant, where the method can comprise: mixing a saccharide-based composition with an alcohol in the presence of an acid catalyst and heating the mixture to a temperature of 100° C. to 250° C. for 2 minutes to 4 hours. In some methods, the saccharide-based composition can comprise a lignocellulosic biomass, a cellulose, a polysaccharide, or a sugar. In some embodiments, the alcohol can be an organic diol. For some methods, the organic diol can be a 1,4-butanediol. In some embodiments, the acid catalyst is sulfuric acid. For some methods, the step of heating can comprise heating the mixture to a temperature of 120° C. to 160° C. for 10 minutes to 45 minutes. With some embodiments, the step of heating comprises heating the mixture to a temperature of about 150° C. for about 20 minutes to about 25 minutes. In some methods, the step of mixing a saccharide-based composition with an alcohol in the presence of an acid catalyst can comprise mixing a mass ratio of about 1:2 to about 10:1 saccharide-based composition to alcohol in the presence of about 0.1 wt. % to about 5 wt. % acid catalyst. For some methods, the step of mixing a saccharide-based composition with an alcohol in the presence of an acid catalyst can comprise mixing a mass ratio of about 2.5:1 by mass saccharide-based composition to alcohol in the presence of about 0.5 wt. % acid catalyst. Some embodiments also describe a fermentation stimulant, where the stimulant is made by the aforedescribed methods. These embodiments and more are described in further detail below.

### BRIEF DESCRIPTION OF THE DRAWINGS

FIG. 1 is a process diagram depicting one possible embodiment of a method for improving fermentation; the optional steps are shown as dashed boxes and are present depending on the composition of the fermentation precursor.

FIG. 2 is a process diagram depicting one possible embodiment of a method for synthesizing a fermentation stimulant, optional steps are shown as dashed boxes.

FIG. 3 is a mass spectroscopy plot verifying the presence of the fermentation stimulant.

FIG. 4 is a plot showing fermentation ethanol yields of xylose fermenting yeast M11205 as percent of theoretical maximum for fermentation of sugar control containing the same amount of initial sugars as CELF hydrolyzate with incremental addition of Fermentation Stimulant (FS-2) over the range of 0% to 33% by volume, where the sugar concentrations in all flasks were identical.

FIG. 5 is a chart showing one-day M11205 fermentation ethanol yields as percent of theoretical maximum from fermentation of a sugar solution containing the same amount of initial sugars as CELF hydrolyzate with additions of 2% FS-2 and 10% Tween 20 in comparison to sugar control, where the sugar concentrations in all flasks were identical.

FIG. 6 is a plot showing a comparison of M11205 fermentation ethanol yields as percent of theoretical maximum from fermentation of a sugar control solution with 50 g/L xylose, and a 50 g/L xylose solution with additional 10% FS-1, where the sugar concentrations in both runs in all flasks were identical.

FIG. 7 is a graph depicting the comparison of M11205 fermentation ethanol yields as percent of theoretical maximum from fermentation of a sugar control solution with glucose and xylose concentrations similar to those in CELF hydrolyzate, with the experimental solution adding an additional 1:1 THF:water and 0.5 wt. % H₂SO₄, which was neutralized with ammonia and boiled at 75° C. to remove THF before combining, where the sugar concentrations in all the flasks compared were identical.

FIG. 8 is a plot presenting a comparison of M11205 fermentation ethanol yields as percent of theoretical maximum from fermentation of a sugar control solution with 50 g/L xylose, where the experimental solution added an additional 10% vol. dilute sulfuric acid (DSA) hydrolyzate, where the sugar concentrations all the flasks compared were identical.

FIG. 9 is a plot depicting xylose concentrations (left axis) and M11205 fermentation ethanol yields as percent of theoretical maximum (right axis) resulting from fermentations of a 100 g/L xylose control and 100 g/L xylose to which had been added a 10% concentration of FS-2, where the sugar concentrations all the flasks compared were identical.

FIG. 10 is a plot of the M11205 fermentation ethanol yields with added 2%, 15%, or 33% FS-1 as percent of theoretical maximum from fermentation as compared to a glucose-xylose control (no addition).

FIG. 11 is a plot of the M11205 fermentation ethanol yields with added 2%, 15%, or 33% CE-1 as percent of theoretical maximum from fermentation as compared to a glucose-xylose control (no addition).

FIG. 12 is a plot of the M11205 fermentation ethanol yields with added 2%, 15%, or 33% CE-2 as percent of theoretical maximum from fermentation as compared to a glucose-xylose control (no addition).

FIG. 13 is a plot of the M11205 fermentation ethanol yields with added 1,4-butanediol as compared to a glucose-xylose control (no addition).

FIG. 14 is a plot of the M11205 fermentation ethanol yields with added 1,4-butanediol for various higher glucose-xylose concentrations.

FIG. 15 is a plot of percentage of theoretical K. marxianus versus time in accordance with an exemplary embodiment.

### DETAILED DESCRIPTION

The details of one or more embodiments of the presently disclosed subject matter are set forth in the accompanying description below. Other features, objects, and advantages of the presently disclosed subject matter will be apparent from the specification, drawings, and claims.

As used herein, the term “alkyl” refers to a moiety comprising carbon, hydrogen, and containing no double or triple bonds. An alkyl can be linear, branched, cyclic or any combination thereof. Examples include methyl, ethyl, propyl, isopropyl, cyclopropyl, n-butyl, iso-butyl, tert-butyl, cyclo-butyl, pentyl isomers, cyclo-pentyl, and the like. An alkyl and be substituted or unsubstituted, where when substituted the hydrogen is replaced by a substituting group. For example, hydroxide may be substituted on the end of an alkyl to form a hydroxy-alkyl moiety.

As used herein, the term “C_{X-Y}” or “C_{X}-C_{Y}” refers to a carbon chain having from X to Y carbon atoms. For example, C₁₋₁₀ alkyl includes fully-saturated hydrocarbon chains having 1, 2, 3, 4, 5, 6, 7, 8, 9, or 10 carbon atoms.

This disclosure describes highly-potent microbial stimulating compositions, or fermentation stimulants, for use in fermentation of sugars and sugar sources to alcohols. Some fermentation stimulants can comprise yeast stimulants that can greatly enhance xylose uptake rates and ethanol yields from anaerobic fermentations by microorganisms. The micro-organisms can comprise yeast, such as engineered S. cerevisiae. For some embodiments, the micro-organisms can comprise bacterium. In addition, some stimulants described can also enhance glucose uptake rates and aerobic cell growth in addition to accelerating anaerobic sugar fermentations to ethanol. Also described are methods of producing the fermentation stimulant. In some embodiments, the stimulants can be produced from sugars. Some embodiments also describe an improved method of fermentation using the aforementioned stimulant.

### Enhanced Fermentation Mixture Containing Precursor and Stimulant

Some embodiments can describe an enhanced fermentation mixture. In some mixture embodiments, the mixture can comprise a sugar-containing fermentation precursor and a fermentation stimulant. In some embodiments, the fermentation enhanced can comprise anaerobic fermentation or aerobic fermentation. In some embodiments, the fermentation enhanced can comprise a combination of both aerobic and anaerobic fermentations. In accordance with an exemplary embodiment, the fermentation enhanced be an increased cell mass or fermentation yield, or both an increased cell mass and fermentation yield.

For some mixtures, the fermentation precursor can be formed from organic material or biomass. In some embodiments, the organic material can comprise biomass, such as a biomass derived from corn, soy beans, tubers (for example, potatoes, sweet potatoes), sugarcane, sorghum, cassava, grasses (for example, switchgrass, Miscanthus, wheat, rice, barley, oats, millet, cassava), legumes, wood (e.g., maple, oak, poplar, pine) or other cellulose substrates, such as agricultural wastes (for example, sugarcane bagasse, corn fiber, corn stover, wheat husk, rice husk). Some biomasses can comprise a lignocellulose biomass. In some embodiments, the biomass can comprise Alamo switchgrass or Maple woodchips. In some embodiments, the fermentation precursor can comprise hemicellulose-derived sugars. In some mixtures, the sugar-containing fermentation precursor can comprise glucose such as made from the starch in corn kernels. In some mixtures, the sugar-containing fermentation precursor can comprise xylose. In some mixtures, the sugar-containing fermentation precursor can comprise both glucose and xylose. In some fermentation precursors the mass ratio of xylose to glucose can range from about 0, about 0.5, about 1, about 2, about 4 about 5, about 6, about 6.8, about 7, about 9, about 9 to about 10, or any combination of ranges, such as about 0, about 6.8. In other embodiments, the fermentation precursors can comprise of mass ratio of glucose to xylose can be about 0.

In some embodiments, a fermentation stimulant can comprise a composition that acts as a surfactant to increase sugar transport into the yeast cell. For some embodiments, the composition can comprise a glucoside, such as an alkyl glucoside or an alkyl polyglycoside. In some embodiments, the alkyl glycoside can comprise a hydroxy-C₃₋₈ alkyl glucopyranoside. In some fermentation enhancers, the glucopyranoside can comprise 4-hydroxybutyl glucopyranoside in both α and β anomeric conformations.

The fermentation stimulant can be applied to any microbial fermentation process to improve production either by increasing cell mass or improving product yields. An example in an existing fermentation application would be adding the enhancer at small amounts to corn starch ethanol production fermentation processes to enhance sugar uptake and ethanol yield. Additionally, the stimulant can be incorporated into cane sugar to ethanol fermentations as well as any other existing fermentation types that use glucose, gluco-oligomers, or other sugars such as sucrose. An example of a potential application is the addition of such a stimulant to a fermentation process for the uptake of pentose sugars by engineered S. cerevisiae. As pentose uptake is not native to S. cerevisiae, its uptake and conversion even after genetic engineering of the microorganism is slow. In addition, increased anaerobic fermentation yields can be obtained with K. marxianus. Another potential application can be the addition of such a stimulant to a fermentation process for bioprocessing via the bacterium, such as Clostridium thermocellum. The addition of the stimulant is envisioned to dramatically increase cell pentose sugar uptake to yield highly-accelerated ethanol yields.

### Method of Improving Fermentation Using the Fermentation Stimulant

Some embodiments can comprise a method for improving fermentation by using a stimulant. In some methods, the improvement of fermentation leads to increased ethanol production from fermenting biomass.

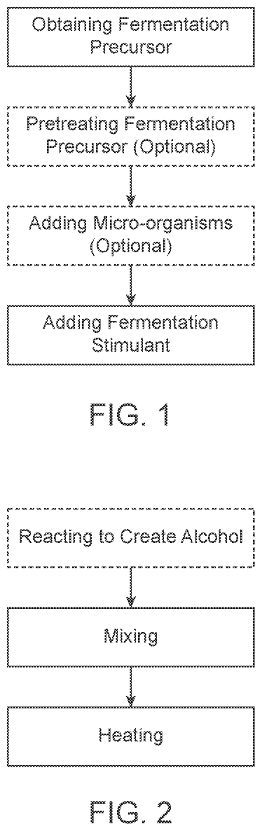

For some embodiments, the method of improving fermentation, as shown in FIG. 1, can comprise: obtaining a fermentation mixture, adding a fermentation stimulant to the fermentation mixture, before fermentation is commenced, and proceeding with fermentation.

In some embodiments, obtaining a fermentation mixture can comprise methods known by those in the art for obtaining a mixture of raw material, such as biomass. In some embodiments, the raw material is ready for fermentation. In some embodiments, obtaining a fermentation mixture can further comprise pretreating biomass using acid-based thermochemical pretreatments or a CELF pretreatment to form a liquid hydrolyzate. In some embodiments, acid-based thermochemical pretreatments can comprise dilute sulfuric acid (DSA) pretreatment. In some embodiments, obtaining a fermentation mixture can additionally comprise adding microorganisms to the fermentation mixture. In some embodiments, the microorganisms can comprise yeast. In some embodiments, the yeast can comprise S. cerevisiae, such as M11205 and D₅A. In some embodiments the micro-organism can comprise a bacterium, such as Clostridium thermocellum.

In some embodiments, the step of adding the fermentation stimulant comprises adding a glucoside to the fermentation mixture. In some methods, the step of adding glucoside comprises adding hydroxy-C₃₋₈ alkyl glucopyranoside, such as 4-hydroxybutyl glucopyranoside, to the fermentation mixture. In some embodiments, adding fermentation stimulant comprises adding between about 0.1% vol., about 0.3% vol., about 0.5% vol., about 1.0% vol., about 2.0% vol., about 3.0% vol., about 5.0% vol., about 10% vol., about 15% vol., about 20% vol., 25% vol., 30% vol., 33% vol., to 35% vol., or any combination thereof. In some methods, the adding fermentation stimulant comprises adding about 2% vol. of fermentation stimulant.

In some methods, commencing fermentation comprises fermenting the mixture using methods known in the art for durations known. In some embodiments, the fermentation mixture is fermented for 24 hours. In some embodiments, the result of the fermentation process is ethanol from biomass.

### Synthesis of the Fermentation Stimulant

Some embodiments include methods of synthesizing a fermentation stimulant. An example embodiment of a synthesis method is presented in FIG. 2. In some embodiments, the method can comprise a modified Co-solvent Enhanced Lignocellulosic Fractionation (CELF) process where the stimulant is synthesized along with other compositions. In some embodiments, the stimulant can be synthesized by a dedicated process and then added to the fermentation before fermentation.

A CELF process typically utilizes a THF:water co-solvent mixture during acid pretreatment. While not wanting to be limited by theory, it is thought that the CELF-like reaction can solubilize some glucose and can hydrolyze some THF to result in the formation of 1,4-butanediol (BDO), an alcohol. Then, it is thought that the combination of BDO and glucose, in the presence of acid can react via a form of Fischer glycosylation to form an alkyl polyglycoside, such as 4-hydroxybutyl glucopyranoside. In accordance with an exemplary embodiment, the ratio of BDO to sugars, for example, glucose, can be from less than about 1% to about 50% or more.

In some embodiments, the method comprises mixing a saccharide-based composition with an alcohol, to produce fermentation enhancer and heating the mixture. In some embodiments, the method can be a one-step reaction by mixing and heating concurrently. In other embodiments, the method can comprise more than one-step, such as mixing and then heating. In some methods, the mixing can be done in the presence of a catalyst. In some embodiments, the catalyst can comprise an acid catalyst, such as sulfuric acid. In some methods, the saccharide-based composition can comprise a lignocellulosic biomass, a cellulose, a polysaccharide, or a sugar. In some embodiments, the sugar can comprise glucose. In some embodiments, the alcohol can comprise an organic diol, such as 1,4-butanediol. In some embodiments, the alcohol can be created as the result of another reaction, such as a byproduct of a CELF-like reaction.

In some embodiments, the mixing step can comprise mixing a saccharide-based composition with an alcohol in the presence of acid catalyst, In some embodiments, the mass ratio of the saccharide-based composition to alcohol can range from about 1:2, about 1.5:1, about 2:1, about 2.5:1, about 3:1, about 4:1, about 5:1, about 7.5:1, to about 10:1, or any combination thereof, such as about 2.5:1.

In some embodiments, the step of mixing can be done with an acid catalyst is between about 0.1 wt. %, about 0.2 wt. %, about 0.3 wt. %, about 0.5 wt. %, about 0.6 wt. %, about 0.75 wt. %, about 1 wt. %, about 2 wt. %, about 3 wt. %, to about 5 wt. %, or any combination thereof, such as about 0.5 wt. % acid catalyst. Where weight percent is based on the saccharide-based composition and alcohol aqueous mixture. In some methods, the step of mixing can comprise mixing in sulfuric acid.

In some embodiments, the heating step can comprise heating to a temperature ranging from about 100° C. to about 250° C., about 110° C. to about 220° C., about 120° C. to about 200° C., about 130° C. to about 175° C., about 140° C. to about 160° C., or any combination thereof, such as about 150° C. or about 160° C. In some mixing steps, the duration of mixing can range from about 2 minutes to 4 hours, about 10 minutes to about 4 hours, about 15 minutes to about 2 hours to about 15 minutes to about 1 hour, from 20 minutes to 30 minutes, or any combination thereof, such as about 25 minutes or about 30 minutes. In some embodiments, the heating step can be at a temperature of about 120° C. to about 160° C. for about 10 minutes to about 45 minutes. In some methods, the heating step can be at a temperature of about 150° C. to about 160° C. for about 20 minutes to about 30 minutes, such as about 150° C. for about 20 to about 25 minutes. The result is a fermentation stimulant. In some embodiments, the fermentation stimulant can be a surfactant that increases substrate uptake during microbial fermentations.

In some methods, the alcohol can be a by-product of reacting glucose and hydrolyzation of THF, specifically the solubilization of glucose and hydrolyzation of THF. In some methods, the organic diol can be a by-product of solubilization of glucose and hydrolyzation of THF. In some methods, the excess THF can be removed from the mixture, such as by boiling the resulting products.

Some embodiments describe stimulant created by mixing a saccharide-based composition with an alcohol in the presence of an acid catalyst and heating the mixture to a temperature of about 100° C. to 250° C. for 2 minutes to 4 hours, for example, about 100° C. to 250° C. for 2 minutes to 2 hours (or 120 minutes). In some embodiments, the saccharide-based composition can comprise glucose. In some embodiments the stimulant can be created by heating the mixture to a temperature of about 100° C. to about 250° C., about 110° C. to about 220° C., about 120° C. to about 200° C., about 130° C. to about 175° C., about 140° C. to about 160° C., or any combination thereof, such as about 150° C. or about 160° C. In some embodiments, the mixture can be heated for about 2 minutes to about 4 hours, about 10 minutes to about 4 hours, about 15 minutes to about 2 hours to about 15 minutes to about 1 hour, from 20 minutes to 30 minutes, or any combination thereof, such as about 25 minutes or about 30 minutes. In some embodiments, the mixture can be heated at a temperature of about 120° C. to about 160° C. for about 10 minutes to about 45 minutes. In some embodiments, the mixture can be heated at a temperature of about 150° C. to about 160° C. for about 20 minutes to about 30 minutes, such as about 150° C. for about 20 minutes to about 25 minutes.

### EMBODIMENTS

The following embodiments are specifically contemplated by this disclosure:

Embodiment 1: An enhanced fermentation mixture, the mixture comprising a sugar-containing fermentation precursor and a fermentation stimulant, the fermentation stimulant being hydroxy-C₃₋₈ alkyl glucopyranoside.
Embodiment 2: The mixture of Embodiment 1, wherein the hydroxy-C₃₋₈ alkyl glucopyranoside comprises a 4-hydroxybutyl glucopyranoside.
Embodiment 3: The mixture of Embodiment 1 or 2, wherein the sugar-containing fermentation precursor comprises a lignocellulose biomass.
Embodiment 4: The mixture of Embodiment 1, 2, or 3, wherein the sugar-containing fermentation precursor comprises a glucose and/or a xylose.
Embodiment 5: The mixture of Embodiment 1, 2, 3, or 4, wherein the mass ratio of xylose to glucose can vary from 0 to 7.
Embodiment 6: The mixture of Embodiment 1, 2, or 3, wherein the sugar-containing fermentation precursor comprises xylose.
Embodiment 7: A method of improving fermentation, the method comprising: obtaining a fermentation mixture, adding a fermentation stimulant to the fermentation mixture before fermentation is commenced, and proceeding with fermentation, where adding the fermentation stimulant comprises adding a glucoside to the fermentation mixture.
Embodiment 8: The method of Embodiment 7, where the step of adding glucoside comprises adding hydroxy-C₃₋₈ alkyl glucopyranoside to the fermentation mixture.
Embodiment 9: The method of Embodiment 7 or 8, where the step of adding glucoside comprises adding 4-hydroxybutyl glucopyranoside to the fermentation mixture.
Embodiment 10: The method of Embodiment 7, 8, or 9, where adding the fermentation stimulant comprises adding between 0.1% vol. to 35% vol. of the stimulant to the mixture.
Embodiment 11: The method of Embodiment 7, 8, 9, or 10, where adding the fermentation stimulant comprises adding 2% vol. of the stimulant to the mixture.
Embodiment 12: A method of making a fermentation stimulant, the method comprising: mixing a saccharide-based composition with an alcohol in the presence of an acid catalyst and heating the mixture to a temperature of 100° C. to 250° C. for 2 minutes to 4 hours.
Embodiment 13: The method of Embodiment 12, where the saccharide-based composition can comprise a lignocellulosic biomass, a cellulose, a polysaccharide, or a sugar.
Embodiment 14: The method of Embodiment 12 or 13, where the alcohol is an organic diol.
Embodiment 15: The method of Embodiment 12, 13, or 14, where the organic diol is a 1,4-butanediol.
Embodiment 16: The method of Embodiment 12, 13, 14, or 15, where the acid catalyst is a sulfuric acid.
Embodiment 17: The method of Embodiment 12, 13, 14, 15, or 16, where the step of heating comprises heating the mixture to a temperature of 120° C. to 160° C. for 10 minutes to 45 minutes.
Embodiment 18: The method of Embodiment 12, 13, 14, 15, 16, or 17, where the step of heating comprises heating the mixture to a temperature of 150° C. for 20 minutes to 25 minutes.
Embodiment 19: The method of Embodiment 12, 13, 14, 15, 16, 17, or 18, where the step of mixing a saccharide-based composition with an alcohol in the presence of an acid catalyst comprises mixing a mass ratio of 1:2 to 10:1 saccharide-based composition to alcohol in the presence of 0.1 wt. % to 5 wt. % acid catalyst.
Embodiment 20: The method of Embodiment 12, 13, 14, 15, 16, 17, 18, or 19, where the step of mixing a saccharide-based composition with an alcohol in the presence of an acid catalyst comprises mixing a mass ratio of 2.5:1 by mass saccharide-based composition to alcohol in the presence of 0.5 wt. % acid catalyst.
Embodiment 21: A stimulant created by the method of claim 12.

### EXAMPLES

It has been discovered that embodiments of the fermentation stimulant resulted in enhanced fermentation rates even at nominal concentrations. While not wanting to be limited by theory, the enhanced microbial performance is believed to be due the stimulant acting as a surfactant to increase xylose transport into the microbe cells, resulting in increased fermentation.

All stimulant forming reactions, including synthesis of the fermentation stimulant, were performed in a 1 L Hastelloy Parr® autoclave reactor (236HC Series, Parr Instruments Co., Moline, Ill.) equipped with a double stacked pitch blade impeller rotated at 200 rpm.

### Example 1.1: Synthesis of Fermentation Stimulant

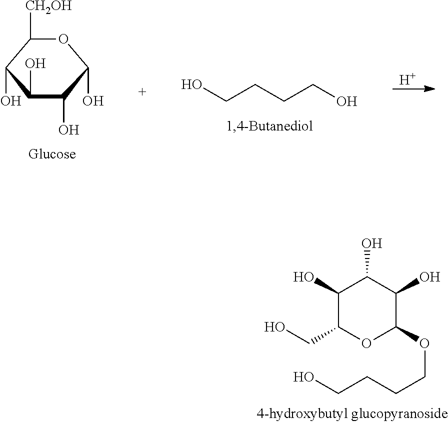

To the autoclave reactor (236HC Series, Parr Instruments Co.) was added 6.25 g/L glucose (aq. sol., Sigma Aldrich, St. Louis, Mo. USA), 2.5 g/L 1-4 butanediol (aq. sol., Sigma Aldrich), and 0.5 wt. % sulfuric acid (Ricca Chemical Company, Arlington, Tex. USA) and the reaction was let go at 150° C. for 25 minutes. Reactions were maintained at temperature (±1° C.) by convective heating with a 4-kW fluidized sand bath (Model SBL-2D, Techne, Princeton, N.J.). Reaction temperature was directly measured using an in-line K-type thermocouple (Omega Engineering Inc., Stamford, Conn.).

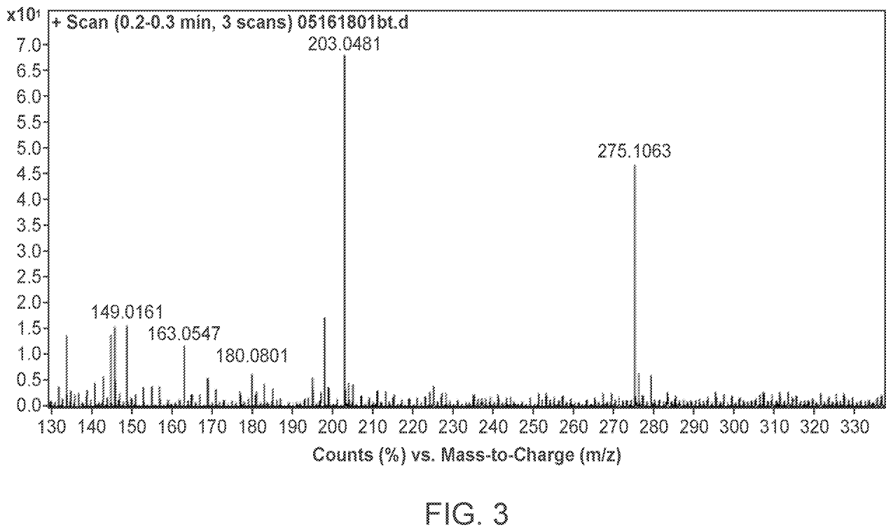

The result was a fermentation stimulant derived from glucose (FS-1), or 4-hydrosbutyl glucopyranoside. Verified by Mass Spectroscopy: calculated for C₁₀O₇H₂₀ (M+Na)⁺ m/z=275.25, found m/z=275.11 (as shown in FIG. 3, noting what appears to be unreacted glucose at m/z=203.04).

### Example 1.2: Synthesis of Fermentation Stimulant Via a Modified CELF Reaction

A modified CELF reaction was performed to synthesize liquid hydrolyzate comprising the fermentation stimulant. Alamo switchgrass (Genera Energy Inc., Vonore, Tenn. USA) was knife-milled to using a Thomas Wiley Laboratory Mill Model 4 (Arthur H. Thomas Company, Philadelphia, Pa.) with a 1 mm particle size interior sieve. To the autoclave reactor was added THF (>99% purity, Fisher Scientific, Pittsburgh, Pa. USA) and water at a volume ratio of 1:1 (or mass ratio of 0.889:1) and 0.5 wt. % (based on liquid mass) sulfuric acid (Ricca Chemical Company, Visalia, Calif. USA) as a catalyst. Then, milled switchgrass (7.5 wt. %) was added to the solution and soaked overnight at 4° C. The reaction was performed at 150° C. for 20 minutes to 25 minutes. Reactions were maintained at temperature (±1° C.) by convective heating with a 4-kW fluidized sand bath (Model SBL-2D, Techne Calibration, Staffordshire UK). Reaction temperature was directly measured using an in-line K-type thermocouple (Omega Engineering Inc., Norwalk, Conn. USA). After the reaction, liquid hydrolyzate was separated from the solid fraction by vacuum filtration at room temperature through glass fiber filter paper (Fisher Scientific, Pittsburgh, Pa. USA). Then, ammonium hydroxide solution (30%, Sigma Aldrich) was slowly added to the liquid hydrolyzate until a pH of 6 was achieved. The pH of the liquid hydrolyzate was measured using an Orion™ Model 91-72 Sure-Flow pH Electrode (ThermoFisher Scientific, Waltham, Mass. USA). The hydrolyzate was then poured into 500 mL flasks and placed in a water bath (Model 14575-12, Cole Palmer, Vernon Hills, Ill.) set at 75° C. in a fume hood, and the hydrolyzate solution was boiled for 8 hours to remove the THF. Hydrolyzate was then filtered through a 0.22 μm sterile filter (Stericup, Millipore Sigma, St Louis, Mo. USA) to separate solid lignin precipitate from the sterile filtrate. The result was a fermentation stimulant derived from switchgrass (FS-2).

### Comparison Example 1.1: Synthesis of Comparison Fermentation Stimulant #1

Comparative Fermentation Stimulants were synthesized to examine whether the 4-hydrosbutyl glucopyranoside was the stimulant. The preparation methods were done in the same manner as Example 1 but except for the following modifications outlined in Table 1.

TABLE 1
Variation of Embodiments for Control Examples.

| Embodiment | Mixture | Temperature | Duration |
|---|---|---|---|
| FS-1 | 6.25 g/L glucose, | 150° C. | 20 min |
| | 4.2 g/L 1,4-butanediol, | | |
| | 0.5 wt. % sulfuric acid | | |
| CE-1 | 43 g/L xylose, | 150° C. | 20 min |
| | 4.2 g/L 1,4-butanediol, | | |
| | 0.5 wt. % sulfuric acid | | |
| CE-2 | 6.5 g/L glucose, | 150° C. | 20 min |
| | 43 g/L xylose, | | |
| | 4.2 g/L 1,4-butanediol, | | |
| | 0.5 wt. % sulfuric acid | | |

Verification of the mass balance for each of FS-1, CE-1, and CE-2 shows the presence of various reactions for each species, as shown in Table 2, and the loss of each reactant species after the reaction.

TABLE 2
Verification Mass Balances After Synthesis Reaction.

| | Glucose | Xylose | 1,4 |
|---|---|---|---|
| | Lost | Lost | butanediol |
| | (% of | (% of | Lost (% |
| Reaction Mixture (before synthesis) | total) | total) | of total) |
| FS-1: glucose + 1,4-butanediol | 18.2 | 0.00 | 4.66 |
| CE-1: xylose + 1,4-butanediol | 0 | 10.71 | 33.09 |
| CE-2: glucose + xylose + 1,4-butanediol | 9.72 | 9.45 | 17.79 |

### Example 2.1: Preparation of the Yeast Stock

Two strains of S. cerevisiae: M11205 and D₅A were tested. To prepare the yeast stock, 1 mL of M11205 or D₅A yeast stock that was frozen at −80° C. was added to 500 mL Erlenmeyer baffled flasks equipped with vent caps (Fisher Scientific) along with 5 mL of 500 g/L glucose, 5 mL of yeast extract, and peptone (100 g/L and 200 g/L) and 39 mL of deionized (DI) water. After 24 hours of incubation for M11205 and 12 hours of for D₅A, the optical density at 600 nm (OD₆₀₀) was measured to determine cell density. Growth times were set to achieve OD₆₀₀ in the range of 6-8 for both strains. The amount of cells to be transferred to anaerobic flasks was determined by the following calculation:

$$Volume from seed flask = \frac{Anaerobic flask volume * 0.5}{Seed flask OD} * ( Number of anaerobic flasks + 1 )$$

The appropriate volume from the seed flask was centrifuged at 2400 rpm for 15 minutes in a benchtop centrifuge (Allegra X15-R, Beckman Coulter, Brea, Calif. USA). The supernatant was decanted and the cells were then resuspended in sterile deionized (DI) water before being centrifuged again. Finally, the cells were resuspended in a volumetric amount of water measured in mL equivalent to the number of anaerobic flasks +1.

### Example 2.2: Benchmarking Stimulant Performance—Glucose Control

To validate the performance of the stimulant, a control group of 50 g/L of glucose (Sigma Aldrich) was anaerobically fermented and compared at cell harvest times.

The control anaerobic fermentations were performed in triplicate in 125 mL flasks with a 50 g working mass that contained glucose, sodium citrate buffer (50 mM, pH 4.8), yeast extract and peptone (10 g/L and 20/L, Becton, respectively; Dickinson and Company, Redlands, Calif. USA), tetracycline (40 mg/L, Sigma Aldrich) as an antimicrobial agent, and yeast inoculum from the seed culture. Empty flasks with bubble traps attached were autoclaved at 121° C. for 35 minutes. Flasks were then cooled and moved into a laminar flow hood (Baker and Baker Ruskinn, Sanford, Me.) for aseptic addition of yeast extract, peptone, citrate buffer, tetracycline, and cell inoculum. 500 μL samples of fermentation liquid were taken at time zero and every 24 hours thereafter. Samples were centrifuged, and the supernatant diluted four times in a glass 2 mL screw top vial (Agilent Technologies, Santa Clara, Calif. USA).

### Example 2.3: Benchmarking Stimulant Performance (FS-2)—Glucose with Stimulant

An experimental group of triplicate anaerobic fermentations was additionally performed using the same method as in Example 2.2 with the exception that 1 mL of FS-2 was additionally added, for a ratio of 1 mL FS-2 per 50 g/L of glucose.

### Example 2.4: Benchmarking Stimulant Performance (FS-2)—Comparison of Glucose Control with Glucose with Stimulant

Liquid samples along with appropriate calibration standards were analyzed by HPLC (Waters Alliance e2695 system equipped with a Bio-Rad Aminex® HPX-87H column and Waters 2414 RI detector, Waters Corporation Milford, Mass. USA) with an eluent (5 mM sulfuric acid, Ricca Chemical) flow rate of 0.6 mL/min.

TABLE 3
Comparison of Cell Density for two Strains of
S. cerevisiae for Glucose and Glucose with the Stimulant.

| | | OD₆₀₀ at |
|---|---|---|
| Strain | Flask Contents | cell harvest* |
| M11205 | Glucose | 7.68 |
| | Glucose +1 mL FS-2 | 9.43 |
| D5A | Glucose | 6.60 |
| | Glucose +1 mL FS-2 | 8.08 |

The results in Table 3 show that addition of as little as 1 mL of FS-2 to a 50 mL seed flask increased OD₆₀₀ significantly for both strains.

### Example 2.5: Stimulant FS-2 Performance in Accelerating Biomass Fermentation Reactions

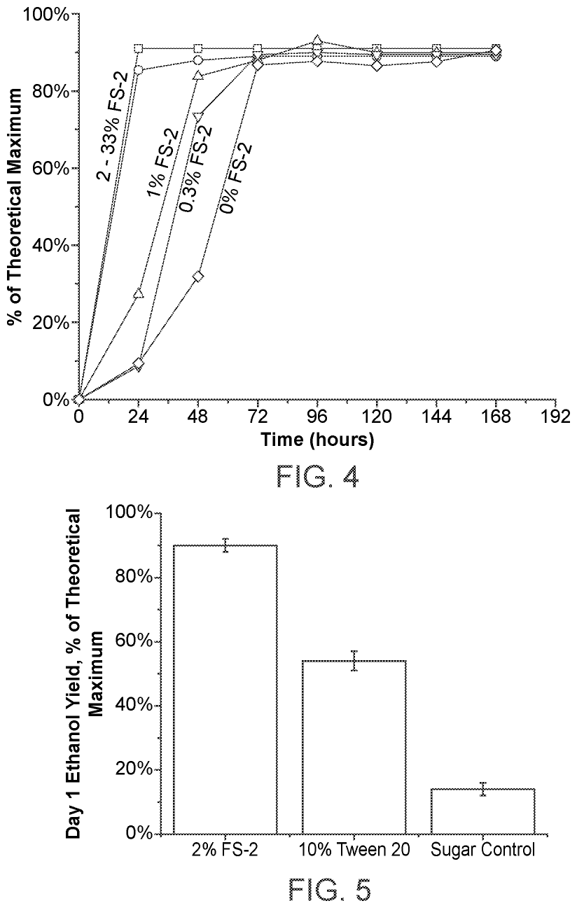

To determine the impact of FS-2 on xylose conversion in conditions like those encountered in a CELF reaction, samples were created using methods similar to Example 2.3, with the exception that the concentrations of sugars and FS-2 was manipulated in the following manner. FS-2 was varied with increasing proportions of FS-2 over the range of 0% to 33% by volume were added to anaerobic M11205 fermentations of sugar solutions for which glucose and xylose concentrations in each flask similar to those found in CELF hydrolyzate was used instead of 50 g/L glucose, for example, 6.5 g/L glucose, 43 g/L xylose (Sigma Aldrich). The results, in FIG. 4, show that a FS-2 concentration as low as 0.3% increased fermentation rates compared to the control sugars without any FS-2 added (labeled 0% FS-2). For FS-2 concentrations in the range of 2% to 33% FS-2, the sugars were completely converted to ethanol within 1 day of anaerobic fermentation. However, at FS-2 concentrations >33%, it was thought that inhibition by lignin-derived phenolics and possibly other compounds in the FS-2 was greater than the stimulation.

### Comparative Example 2.1

To determine the impact of FS-2 versus other known surfactants on xylose conversion in conditions like those encountered in a CELF reaction, samples were created using methods similar to Example 2.3, with the exception that the concentrations of sugars and FS-2 was manipulated in the following manner. Instead of FS-2, Tween 20 at 10% by volume was added to anaerobic M11205 fermentations of sugar solutions for which glucose and xylose concentrations in each flask are similar to those found in CELF hydrolyzate instead of 50 g/L glucose, for example, 6.5 g/L glucose, 43 g/L xylose.

The one-day fermentation yield of the Tween 20 at 10% vol. was compared to 2% vol. FS-2 and a control that contained no surfactant. The results, shown in FIG. 5, depict that the addition of FS-2 at only 2% by volume dramatically increased the fermentation yields as compared to Tween 20, a commercially available surfactant, at a concentration of 10% by volume.

### Example 2.6 Stimulant FS-1 Performance in Accelerating Conversion of Xylose

An experimental group of triplicate anaerobic fermentations was additionally performed with FS-1 to verify that the increased in fermentation yields was due to the stimulant.

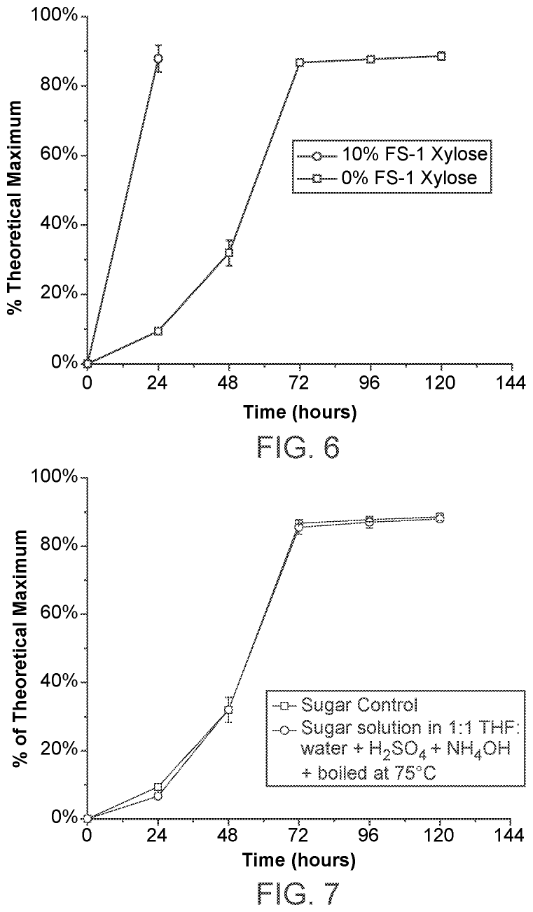

To determine the impact of the stimulant on xylose, samples were created using methods similar to Examples 2.2 and 2.3, with the exception that instead of glucose, 100 g/L of xylose (Sigma Aldrich) was used and for the experimental groups instead of 1 mL of FS-2, 5 mL of FS-1 was used and the yeast M11205 was used. The resulting mixtures were left to anaerobically ferment. As shown in FIG. 6, it was observed that after 24 hours all the sugars in the experimental group were completely converted to ethanol, showing a stimulant effect, where took from 72 hours to 120 hours to achieve a similar effect with glucose alone, demonstrating a clear stimulant effect.

### Example 2.7: Isolation of Enhanced Fermentation Properties to Stimulant

To isolate the enhanced fermentation effect to the stimulant composition, several control and experimental groups were created using methods similar to Examples 2.2 and 2.3 with 50 g/L of xylose (Sigma Aldrich) instead of glucose that tested the effect of other compounds present in a modified-CELF reaction. The experimental groups added the following compounds instead of 1 mL FS-2:
(1) 0.5 wt. % of sulfuric acid and ammonium hydroxide to neutralize to pH 7;
(2) 5 mL of dilute sulfuric acid (DSA) hydrolyzate of switchgrass.

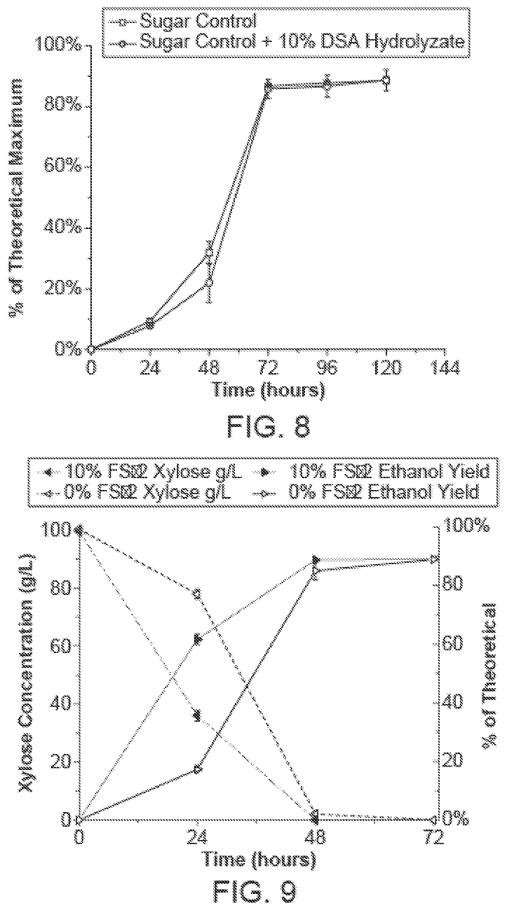

The groups were then anaerobically fermented using M11205 yeast. As shown in FIGS. 7 and 8 for 0.5 wt. % of sulfuric acid and ammonium hydroxide and 5 mL of dilute sulfuric acid (DSA) hydrolyzate, it was observed that both experimental groups did not exhibit a significant difference in fermentation rates or final ethanol yields from the control group.

In addition, the isolated effect of 1,4-butanediol was characterized by creating control and experimental groups using methods similar to Examples 2.2 and 2.3 with the exception that instead of 50 g/L glucose, glucose and xylose was used in concentrations similar to those found in CELF hydrolyzate, for example, 6.5 g/L glucose, 43 g/L xylose. Additionally, for the experimental groups, instead of adding FS-2, 2.5 g/L of 1,4-butanediol (Sigma Aldrich) was used. Both groups were allowed to anaerobically ferment using M11205 yeast for 5 days and then compared to the 5-day theoretical maximum yield.

TABLE 4

| Comparison of 1,4-butanediol Effect on Day-5 Fermentation | |
|---|---|
| Rates as Compared to Theoretical Maximum Ethanol Yield. | |
| Flask BDO | Day 5% of Theoretical |
| Concentration (g/L) | Max. Ethanol Yield [%] |
| 0 | 88.7 |
| 2.5 | 88.8 |

As shown in Table 4, the results indicate 1,4-butanediol does not create the increased fermentation rates. These results support that the increased fermentation performance is due to the stimulant composition.

### Example 2.8: Stimulant FS-2 Performance in Accelerating Conversion of Xylose

To further quantify the impact of the stimulant on xylose, samples were created using methods similar to Examples 2.2 and 2.3, with the exception that the concentrations of sugars and FS-2 was manipulated in the following manner. Instead of glucose, 100 g/L of xylose (Sigma Aldrich) was used and for the experimental groups the amount of FS-2 was varied to 10% by volume and the yeast M11205 was used. The mixtures were anaerobically fermented for a total of 72 hours measuring the xylose g/L and the ethanol yield. The results, shown in FIG. 9, clearly indicate that the stimulant improves the conversion of xylose to ethanol, with more than a three-fold increase in ethanol production in 24 hours.

### Example 2.9: Stimulant Comparison of FS-1, CE-1 and CE-2

To further investigate the properties of the stimulant in comparison to possible other byproducts in the CELF process, FS-1, CE-1, or CE-2 were added to fermentations to determine their relative response. First, each mixture was neutralized to pH of 7 with 30 wt. % ammonium hydroxide (EMD Chemicals Inc., Gibbstown, N.J. USA) following the reaction. Then, for each resulting mixture, glucose, xylose or 1-4 butanediol was added to the mixture to adjust the pre-reacted volume ratios so that all mixtures had the same concentrations of 6.5 g/L glucose, 43 g/L xylose, and 4.2 g/L 1,4 butanediol. Then, the mixtures were sterilized and stored at 4° C. prior to fermentation. Then, the mixtures were fermented using the procedure in Example 2.3 with the exception that each stimulant was added to the fermentation mixture in either 2% by volume, 15% by volume, or 33% by volume, for three test cases for each stimulant, excluding a control case. Examination of the reacted glucose to the total sugars (e.g. glucose and xylose) present in the fermentation, yields the following in Table 5, noting that the total of glucose and xylose present in each fermentation flask after concentration normalization was 2.475 g.

TABLE 5
Amount of Reacted Glucose (from Synthesis) in Fermentation Reaction.

| Reaction Mixture (before synthesis) | 33% vol. | 15% vol. | 2% vol. |
|---|---|---|---|
| FS-1: glucose + 1,4-butanediol | 3.5% | 1.6% | 0.426% |
| CE-1: xylose + 1,4-butanediol | 0% | 0% | 0% |
| CE-2: glucose + xylose + 1,4-butanediol | 3.85% | 1.77% | 0.473% |

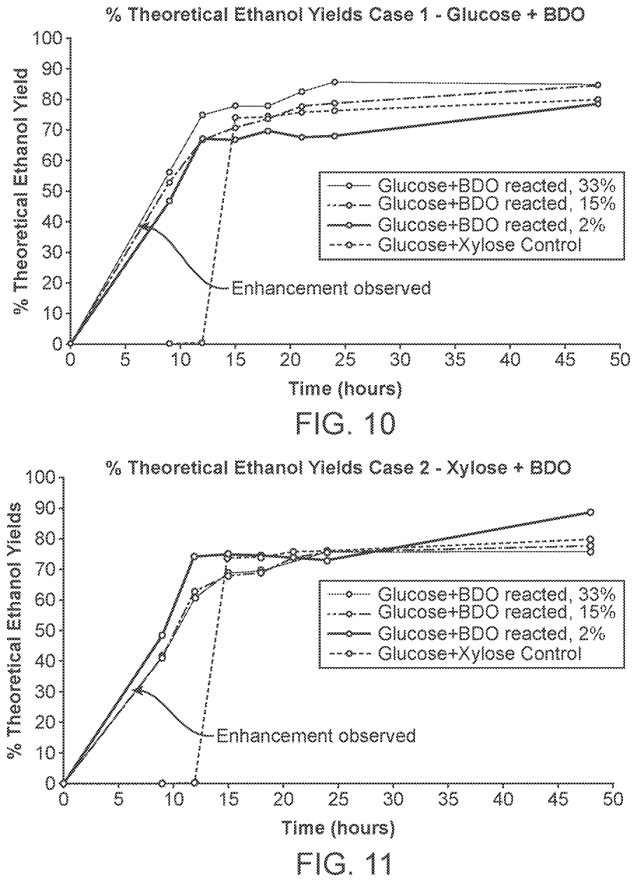

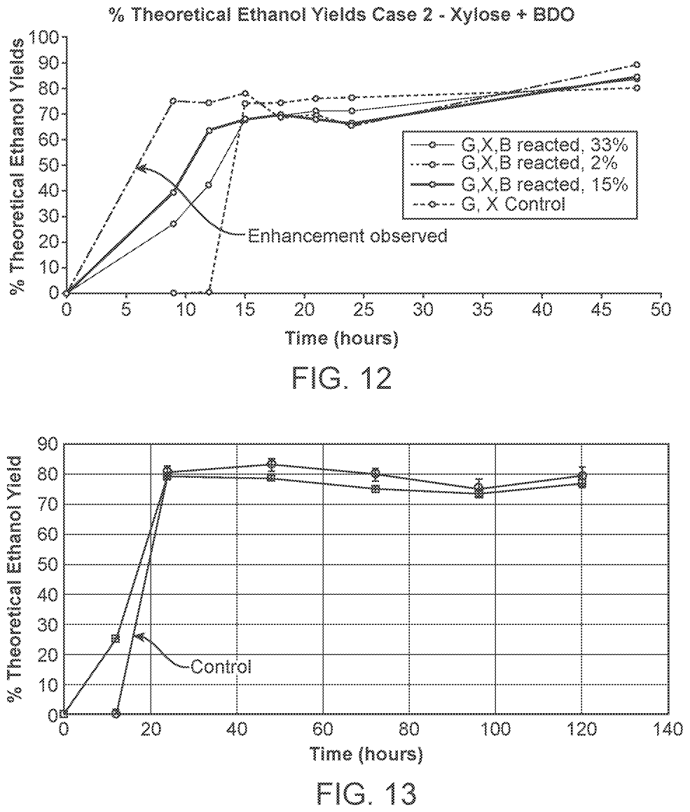

The results are shown in FIG. 10 for FS-1, FIG. 11 for CE-1 and FIG. 12 for CE-2 respectively. When the reactions were compared, it was observed that FS-1 had a distinct enhancement for ethanol yield as well as the elimination of the lag phase.

### Comparative Example 2.2: Stimulant Comparison of FS-1, CE-1 and CE-2

To verify that the stimulant effect was not from 1,4-butanediol, fermentation was conducted following the procedure outlined in Example 2.2 with the exception that 0.21 g or 4.2 g/L of 1,4-butanediol (Sigma Aldrich) was added to the fermentation mixture. The results, shown in FIG. 13, compare a mixture with the addition to a control without the addition and indicate that 1,4-butanediol is not itself a contributor to the stimulant properties.

Further investigation at higher sugar concentrations was done by adding 4.2 g/L of 1,4-butanediol (Sigma Aldrich) where the mixture concentrations before the addition of 1,4-butanediol are depicted in Table 6.

TABLE 6
Variation of Initial Reaction Mixtures Before 1,4-butanediol
Addition to Further Verify 1,4-butanediol Contribution.

| Reaction Mixture | Starting Concentrations (g/L) | | |
|---|---|---|---|
| (before synthesis) | Glucose | Xylose | 1,4-butanediol |
| Example 2.2.1 | 6.5 | 43 | 0 |
| Example 2.2.2 | 6.5 | 43 | 4.2 |
| Example 2.2.3 | 13.5 | 86 | 0 |
| Example 2.2.4 | 19.5 | 129 | 0 |
| Example 2.2.5 | 32.5 | 215 | 0 |

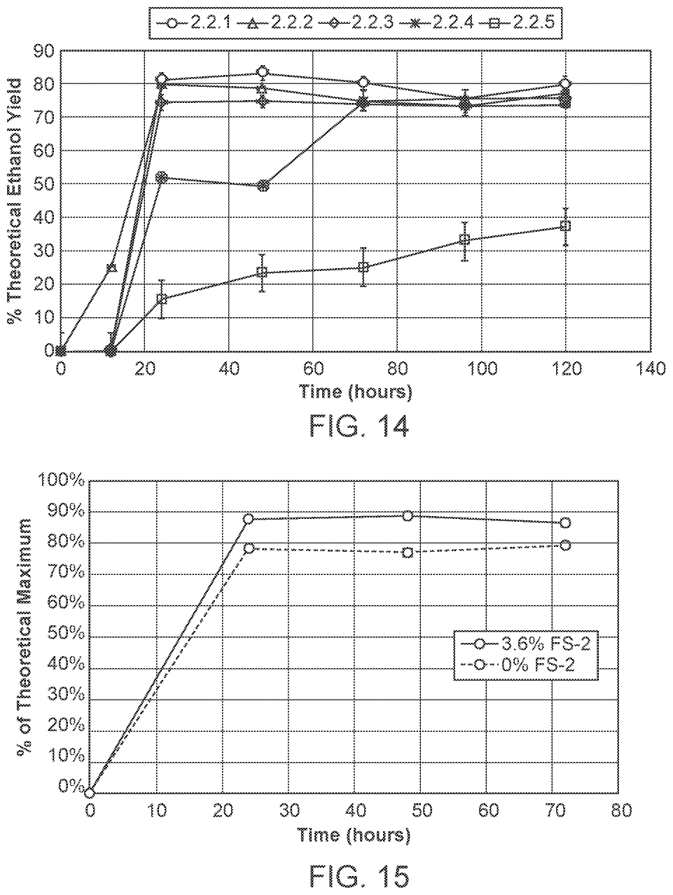

The results, shown in FIG. 14, also show no distinct contribution for enhancement from 1,4-butanediol, further indicating the role of FS-1 or FS-2 as a stimulant.

### Example 2.10: Promoting Growth of K. marxianus

In accordance with another exemplary embodiment, the promotion of growth of K. marxianus was explored. The results of the promotion of growth of K. marxianus is shown in Table 7, and shown in FIG. 15.

TABLE 7
Promoting Growth of K. marxianus.

| | Average % Theoretical Yield | | | |
|---|---|---|---|---|
| Sample | 0 | 24 | 48 | 72 |
| 0% FS-2 | 0% | 78% | 77% | 79% |
| 3.6% FS-2 | 0% | 88% | 89% | 87% |
| | Standard Deviation | | | |
| Sample | 0 | 24 | 48 | 72 |
| 0% FS-2 | 0% | 1% | 1% | 1% |
| 3.6% FS-2 | 0% | 0% | 1% | 1% |

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed considering the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided herein is intended merely to better illuminate the invention and does not pose a limitation on the scope of the invention otherwise claimed. No language in the specification should be construed as indicating any non-claimed element essential to the practice ant of the embodiments disclosed in the present disclosure.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is to be understood that the embodiments of the invention disclosed herein are illustrative of the principles of the present invention. It should be understood that the disclosed subject matter is in no way limited to a particular methodology, protocol, and/or reagent, etc., as described herein. Various modifications or changes to or alternative configurations of the disclosed subject matter can be made in accordance with the teachings herein without departing from the spirit of the present specification. Accordingly, the present invention is not limited to that precisely as shown and described.

What is claimed is:

1. An enhanced fermentation mixture, the mixture comprising:
a sugar-containing fermentation precursor; and
a fermentation stimulant, wherein the fermentation stimulant is a hydroxy-C₃₋₈ alkyl glucopyranoside.

2. The mixture of claim 1, wherein the hydroxy-C₃₋₈ alkyl glucopyranoside comprises a 4-hydroxybutyl glucopyranoside.

3. The mixture of claim 1, wherein the sugar-containing fermentation precursor comprises a lignocellulose biomass.

4. The mixture of claim 1, wherein the sugar-containing fermentation precursor comprises a glucose and/or a xylose.

5. The mixture of claim 4, comprising:
a mass ratio of xylose to glucose from 0 to 7.

6. The mixture of claim 1, wherein the sugar-containing fermentation precursor is xylose.

7. A method of improving fermentation, the method comprising:
obtaining a fermentation mixture;
adding a fermentation stimulant to the fermentation mixture before fermentation is commenced; and
proceeding with fermentation, wherein the adding of the fermentation stimulant comprises adding a glucoside to the fermentation mixture.

8. The method of claim 7, wherein the step of adding glucoside comprises:
adding hydroxy-C₃₋₈ alkyl glucopyranoside to the fermentation mixture.

9. The method of claim 7, wherein the step of adding glucoside comprises:
adding 4-hydroxybutyl glucopyranoside to the fermentation mixture.

10. The method of claim 7, wherein the adding of the fermentation stimulant comprises:
adding between 0.1% vol. to 35% vol. of the stimulant to the mixture.

11. The method of claim 10, wherein the adding of the fermentation stimulant comprises:
adding 2% vol. of the stimulant to the mixture.

12. A method of making a fermentation stimulant, the method comprising:
mixing a saccharide-based composition with an alcohol in the presence of an acid catalyst; and
heating the mixture to a temperature of 100° C. to 250° C. for 2 minutes to 4 hours.

13. The method of claim 12, wherein the saccharide-based composition comprises:
a lignocellulosic biomass, a cellulose, a polysaccharide, or a sugar.

14. The method of claim 12, wherein the alcohol is an organic diol.

15. The method of claim 14, wherein the organic diol is a 1,4-butanediol.

16. The method of claim 12, wherein the acid catalyst is a sulfuric acid.

17. The method of claim 12, wherein the heating comprises:
heating the mixture to a temperature of 120° C. to 160° C. for 10 minutes to 45 minutes.

18. The method of claim 12, wherein the heating comprises:
heating the mixture to a temperature of 150° C. for 20 minutes to 25 minutes.

19. The method of claim 12, where the mixing of the saccharide-based composition with the alcohol in the presence of an acid catalyst comprises:
mixing a mass ratio of 1:2 to 10:1 saccharide-based composition to alcohol in the presence of 0.1 wt. % to 5 wt. % acid catalyst.

20. The method of claim 12, wherein the mixing of the saccharide-based composition with an alcohol in the presence of an acid catalyst comprises:
mixing a mass ratio of 2.5:1 by mass saccharide-based composition to alcohol in the presence of 0.5 wt. % acid catalyst.

21. A fermentation stimulant created by the method of claim 12.
